(19) United States
(12) Patent Application Publication
(10) Pub. No.: US 2022/0105358 A1
(43) Pub. Date: Apr. 7, 2022

(54) OPTOELECTRONIC RADIATION DEVICE

(71) Applicant: OSRAM Opto Semiconductors GmbH, Regensburg (DE)

(72) Inventors: Alexander WILM, Regensburg (DE); Markus HOFMANN, Bad Abbach (DE); Rainer HUBER, Bad Abbach (DE); Stefan GROETSCH, Bad Abbach (DE); Stefan LORENZ, Schwarzenbruck (DE); Stefan MORGOTT, Pentling (DE)

(21) Appl. No.: 17/426,450

(22) PCT Filed: Jan. 29, 2020
(86) PCT No.: PCT/EP2020/052120

§ 371 Date: Jul. 28, 2021

(30) Foreign Application Priority Data
Jan. 31, 2019 (DE) 10 2019 102 508.7

(51) Int. Cl. A61N 5/06; F21V 9/40; F21V 23/04; B60Q 3/70

(52) U.S. Cl. CPC A61N 5/0613; F21V 9/40; F21Y 2113/10; F21V 23/0457; B60Q 3/70; F21V 23/0471

(57) ABSTRACT

Optoelectronic radiation device, in particular for generating and emitting health-promoting and regenerative radiation, comprising: at least one or more optoelectronic radiation sources configured to generate infrared radiation, and at least one optoelectronic radiation source for generating white light, wherein the infrared radiation is in an infrared spectral range between 600 nm and 900 nm.

This patent application claims priority to German application DE 10 2019 102 508.7, the disclosure content of which is hereby incorporated by reference.

The present invention relates to an optoelectronic radiation device, in particular for generating and emitting health-promoting optical radiation.

Technologies in lighting technology are currently being developed primarily with regard to the aspects of luminous efficacy and cost pressure. In order to achieve a high luminous efficacy of a light source, such as a white LED (from light emitting diode), areas without a contribution to the perceived brightness, such as spectral radiation components in the ultraviolet and infrared wavelength range, are excluded from the light generation, so that the focus is on the generation of light with wavelengths in the range between for example 440 nm and 630 nm. In particular, the aim can be to achieve the highest possible congruence with a known brightness sensitivity curve, also known as the V-λ-curve. The brightness sensitivity curve describes the spectral brightness sensitivity of the human eye in daylight.

However, research results show that spectral components of optical radiation that are not visible to the eye also have a regenerative effect on the eye and the human body. For example, the scientific publications by Rizalyn Albarracin et al.: Photobiomodulation Protects the Retina from Light-Induced Photoreceptor Degeneration, Ophthalmology & Visual Science, May 2011, Vol. 52, No. 6 and Joshua A. Chu-Tan et al: Efficacy of 670 nm Light Therapy to Protect against Photoreceptor Cell Death Is Dependent on the Severity of Damage, International Journal of Photoenergy Volume 2016, that infrared radiation has a protective and regenerative effect on cell structures. A list of other scientific publications is given below:
Rizalyn Albarracin et al: Photobiomodulation Protects the Retina from Light-Induced Photoreceptor Degeneration, Ophthalmology & Visual Science, May 2011, Vol. 52, No. 6.
Joshua A. Chu-Tan et al: Efficacy of 670 nm Light Therapy to Protect against Photoreceptor Cell Death Is Dependent on the Severity of Damage, International Journal of Photoenergy Volume 2016.
Shinichiro Fuma et al: Photobiomodulation with 670 nm light increased phagocytosis in human retinal pigment epithelial cells, Molecular Vision 2015; 21:883-892.
Boris Ivandic et al: Low-Level Laser Therapy Improves Vision in Patients with Age-Related Macular Degeneration, Photomedicine and Laser Surgery Volume 26, Number 3, 2008.
T. I. Karu et al.: Exact Action Spectra for Cellular Responses Relevant to Phototherapy, Photomedicine and Laser Surgery Volume 23, Number 4, 2005.
Boris T. Ivandic et al: Low-Level Laser Therapy Improves Vision in Patients with Age-Related Macular Degeneration, Photomedicine and Laser Surgery Volume 26, Number 3, 2008.
Graham F. Merry et al: Photobiomodulation reduces drusen volume and improves visual acuity and contrast sensitivity in dry age-related macular degeneration, Acta Ophthalmologica 2017.

It is an object of the present invention to provide an optoelectronic radiation device capable of generating and emitting optical radiation, which can contribute in an improved manner to enhancing well-being and promoting health.

The object is solved by an optoelectronic radiation device having the features of claim 1. Preferred embodiments and further developments of the invention are given in the dependent claims.

An optoelectronic radiation device according to the invention, which is provided in particular for generating and emitting health-promoting optical radiation, comprises at least one radiation source or a plurality of optoelectronic radiation sources which is/are designed to generate infrared radiation, and at least one optoelectronic radiation source for generating white light, the infrared radiation being in an infrared spectral range between 600 nm and 900 nm.

The infrared spectral range may be continuous or non-continuous. The radiation source for generating white light may be provided in addition to the radiation source(s) for generating infrared radiation. A radiation source may also perform a dual function and serve to generate both visible and infrared radiation.

Spectral components may be present at at least one and preferably at several or all of the following wavelengths:

λ1=670 nm±10 nm,

λ2=760 nm±10 nm,

λ3=780 nm±10 nm and

λ4=830 nm±10 nm.

The infrared radiation may also comprise spectral components at at least one and preferably at several or all of the following wavelengths:

λ5=620 nm±10 nm,

λ6=675 nm±10 nm,

λ7=760 nm±10 nm and

λ8=830 nm±10 nm.

The deviations of ±10 nm can also be wider, e.g. ±30 nm.

According to scientific findings, an improvement of health promotion and regeneration of cell structures can be achieved by the radiation of infrared radiation in the infrared wavelength range, for example at one or more of the mentioned wavelengths λ1 to λ8, if a person is exposed to this light for a sufficiently long period of time. The improvement in health promotion and regeneration of cell structures results in particular in comparison with radiation sources that are optimized only for the radiation of visible light in the visible spectral range, for example between 440 nm and 630 nm.

One, more, or all of the mentioned wavelengths may comprise a local maximum in the infrared emission spectrum of the radiation device.

Optoelectronic radiation sources for generating infrared radiation, in particular at one or more of the mentioned wavelengths, can be manufactured at low cost. Embodiments of optoelectronic radiation devices according to the invention can therefore be realized cost-effectively.

An optoelectronic radiation device according to the invention may comprise a separate optoelectronic radiation source for generating infrared radiation at a predetermined wavelength, in particular for one, several or each of said wavelengths λ1 to λ8. The optoelectronic radiation source may thus comprise, for example, four optoelectronic radiation sources, in particular respective LEDs or LED chips, each light source being configured to generate radiation of a predetermined wavelength, for example at one of said wavelengths λ1 to λ4 or λ5 to λ8.

Embodiments may be provided in which not all four wavelengths λ1 to λ4 or λ5 to λ8 are generated, but only a selection of said wavelengths. The optoelectronic radiation device can then have the corresponding number of optoelectronic radiation sources, wherein each of the optoelectronic radiation sources can be configured to generate and emit exactly one of the wavelengths λ1 to λ8. In this context, the respective radiation source can also be configured to generate a broad spectrum containing the respective wavelength. The radiation source can thus be configured either as a narrow-band emitter or as a broad-band emitter.

It may be provided that each optoelectronic radiation source is arranged side by side on a plane, in particular on a substrate or carrier. Each radiation source can comprise its own enclosure. Thus, for example, a multi-LED module can be formed.

It can be provided that each optoelectronic radiation source is arranged in an associated, own cavity of a housing of the optoelectronic radiation device. Each cavity thereby comprises a bottom and lateral walls. Typically, a respective radiation source is arranged on the bottom of the cavity. On the upper side of the cavity facing away from the bottom, the cavity is open or closed by means of a window. The window is at least partially transparent to the radiation generated by the radiation source. Placing the radiation source in separate cavities can be advantageous in order to avoid interactions (“cross-talk”) between the radiation sources.

In other embodiments of an optoelectronic radiation device according to the invention, at least two optoelectronic radiation sources may also be arranged in the same cavity. A compact design can thus be realized.

It may be provided that at least one radiation source is intrinsically designed to generate infrared radiation, in particular at at least one of the mentioned wavelengths. Thus, the infrared radiation may be generated within the radiation source. For example, the infrared radiation may be generated within an active layer if the radiation source is an LED.

It may also be provided that at least one radiation source is intrinsically designed to generate radiation in a spectral range outside the infrared spectral range, in particular outside the wavelengths λ1 to λ4 and/or the wavelengths λ5 to λ8, and that at least one converter is provided to convert the radiation at least partially into radiation having spectral components in the infrared spectral range, in particular at at least one wavelength λ1 to λ4 or λ5 to λ8. The infrared radiation, for example, at one or more of the mentioned wavelengths can be generated, for example, by means of a suitable conversion material by conversion from blue light generated, for example, by means of an LED. A relatively broad, continuous infrared spectrum comprising one or more of said wavelengths λ1 to λ8 can also be generated. For example, a broad infrared spectrum can be generated by converter solutions pumped with ultraviolet radiation.

It is also possible to generate infrared radiation at one or more of the mentioned wavelengths by optoelectronic radiation sources that have so-called quantum dots. The quantum dots can be manufactured with respect to a desired emission wavelength.

The optoelectronic radiation source for generating white visible light may be used as an illumination device. The radiation source for generating white radiation may be located distant from the radiation source(s) for generating infrared radiation.

The radiation source for generating white light can be arranged in a separate cavity of a housing of the optoelectronic radiation device. An undesired interaction between the white light radiation source and the infrared radiation source or sources can thus be avoided.

However, it may also be provided that the radiation source for generating white light and at least one radiation source for generating infrared radiation are arranged in a common cavity of the housing of the optoelectronic radiation device. This makes it possible, for example, to use the radiation source for generating white light also for generating infrared radiation, in particular at at least one of said infrared wavelengths λ1 to λ8, in particular in combination with a converter material. The converter material may comprise a first converter that converts radiation provided by the radiation source, for example blue or ultraviolet radiation, into white light. The converter material may comprise a second converter that converts radiation provided by the radiation source into infrared radiation, particularly at any of said wavelengths. The first converter and the second converter may be intermixed. The first converter and the second converter may be arranged in a common matrix material.

Embodiments of optoelectronic radiation devices according to the invention may comprise at least one sensor for detecting infrared radiation, in particular at at least one of the mentioned wavelengths λ1 to λ8. Since dose often plays a decisive role in biology, it may be advantageous to integrate such a sensor into an optoelectronic radiation device, in particular to measure the generated radiation dose and to provide the measured data for further processing.

A sensor for detecting infrared radiation, especially at one of the wavelengths λ1 to λ8, can be realized, for example, by operating an LED capable of emitting radiation at the wavelength to be detected in the reverse direction.

A control system can be provided, which is configured to control at least one of the optoelectronic radiation sources for generating infrared radiation as a function of a signal generated by means of the sensor. The control can be set, for example, to emit a specific dose of radiation in a specific unit of time.

The sensor may be arranged in a cavity of a housing of the radiation device. The arrangement of the sensor in its own separate cavity may be advantageous if an undesired influence of the sensor by one of the optoelectronic radiation sources is to be avoided. However, it can also be provided that the sensor is arranged in the same cavity as at least one of the optoelectronic radiation sources for generating infrared radiation. This means that no additional installation space needs to be provided for the sensor.

Each optoelectronic radiation source can be connected to an associated, separate control unit and supplied with electricity via this and optionally also controlled.

For the power supply of the optoelectronic radiation sources, it can also be provided that at least two and preferably all optoelectronic radiation sources for the generation of infrared radiation are electrically connected in series. This allows the current through the optoelectronic radiation sources to be kept constant. If this is not possible, or if the radiation sources, for example in the form of LED chips, are significantly different, a combination of serial and parallel connection may also be possible.

In particular, it can be provided that at least one and preferably all radiation sources, which are configured to generate infrared light, are electrically connected in parallel to at least one radiation source, which is provided for the generation of white light. This is particularly advantageous if the radiation sources are significantly different in terms of their electrical current consumption.

An electrical current control element, such as a series resistor, linear regulator or current mirror, can keep the current through the different radiation sources constant. It may be useful to adjust the electrical voltages of the at least one radiation source used to generate white light, which may be a blue LED, for example, and the at least one radiation source used to generate the infrared radiation by connecting them in series and, if necessary, by inserting current control elements.

The invention also relates to an optoelectronic radiation device, which is provided in particular for generating and emitting health-promoting radiation, and which comprises at least one or more optoelectronic radiation sources for generating infrared radiation and at least one optoelectronic radiation source for generating white light, wherein the infrared radiation comprises, for example, a continuous spectrum comprising at least one and preferably several or all of the following wavelengths:

λ1=670 nm±10 nm or ±30 nm,

λ2=760 nm±10 nm or ±30 nm,

λ3=780 nm±10 nm or ±30 nm, and

λ4=830 nm±10 nm or ±30 nm.

The same applies to the wavelengths λ5 to λ8 or another selection of wavelengths, e.g. from the wavelengths λ1 to λ8.

The invention also relates to an electronic device, such as a cell phone, a tablet, a display screen or a television, wherein the electronic device comprises a display and at least one optoelectronic radiation device according to the invention, and wherein the optoelectronic radiation device is integrated in the display, in particular directly within the display field or in the frame of the display. Due to a normally relatively long dwell time of a user and the relatively predictable viewing scenarios in which a user normally looks at a display, such devices are well suited for irradiation by infrared radiation.

The radiation sources of the optoelectronic radiation device, which are used to generate infrared radiation, can be LEDs in particular. Such IR LEDs can be arranged as “direct backlight” or “edge lit backlight” to the regular LEDs in the display.

Displays used in an electronic device may regularly have an edge filter in the red region. However, such an edge filter usually has no limitation with respect to infrared radiation. Therefore, infrared radiation is not filtered out. Thus, when LEDs are arranged to generate infrared radiation, such an edge filter can normally be retained.

In particular, when using an LCD display, a special IR pixel could also be introduced which, in addition to the regularly existing RGB pixels, allows the generated radiation to pass through and be controlled. The IR pixel could be coupled with a red component, for example by using an additional LED emitting in the red wavelength range, thus avoiding color shift of an image in an improved way.

In some embodiments of the electronic device according to the invention, the optoelectronic radiation device according to the invention with at least one radiation source for generating infrared radiation at at least one of said wavelengths λ1 to λ8 may be arranged in the display frame. For example, an IR LED could be arranged together with a flashlight LED on the side of the display frame of a tablet or smartphone facing the user. This could result in irradiation of the viewer's eye with infrared radiation.

A proximity sensor could also be arranged in the frame of the display. Proximity sensors of this type may already be present in smartphones or tablets, for example. The proximity sensor could be expanded to the necessary powers and wavelength ranges, in particular for emitting at least one of the aforementioned wavelengths λ1 to λ8, so that it could additionally fulfill an irradiation function by emitting significantly more radiation in the infrared wavelength range than would be necessary for distance measurement itself. In combination with an additional position sensor, which can also be housed as standard in the frame of a display, e.g. in a smartphone, a radiation dose can be determined and, if necessary, controlled.

The invention also relates to a motor vehicle having an interior and at least one optoelectronic radiation device according to the invention, the latter being arranged in the interior or at least for illuminating or irradiating the interior.

The lighting provided as standard in a vehicle interior can, for example, additionally comprise at least one radiation device according to the invention which emits infrared radiation. This can also be configured in such a way that it can additionally emit white light. Thus, such a radiation device can, for example, replace or at least supplement conventional lighting in the vehicle interior. Particularly in an autonomous vehicle, the interior can therefore be used as a regeneration cell.

The optoelectronic radiation device can, for example, be arranged in an armature and/or a display element. Infrared radiation can thus emerge from the armature or a display element and act on a vehicle occupant.

An optoelectronic radiation device according to the invention can be arranged in such a way that its emitted infrared radiation can be coupled into the windshield and/or windows of the vehicle and then coupled out toward the interior.

An optoelectronic radiation device according to the invention can be arranged in a display, such as a direct-view display or a head-up display. The radiation sources for generating infrared radiation can be formed, for example, by micro-LEDs that emit in the infrared spectral range. Such LEDs can be installed together with RGB pixels used for imaging and used to generate infrared radiation.

The invention further relates to a projector comprising at least one imaging device and an optical system for projecting an image provided by the imaging device into an image plane, in particular on a screen or the like, the projector further comprising an optoelectronic radiation device according to the invention for providing infrared light.

The infrared radiation can be projected into the image plane by means of the optics. In this case, a feed to the optics can be independent of the imaging device. However, the radiation device for generating the infrared light may also be integrated into the imaging device.

The invention also relates to electronic glasses, in particular AR glasses or VR glasses, in which at least one optoelectronic radiation device according to the invention is arranged in such a way that infrared radiation emitted by it can reach at least one eye of the user of the glasses. AR stands for “augmented reality” and VR stands for “virtual reality”.

It would also be possible to embed an optoelectronic radiation device according to the invention in a contact lens. This could then be charged and/or operated by means of an air interface (“wireless”).

Optoelectronic radiation devices according to the invention could also be integrated into other devices through which irradiation of a user can be achieved. Such devices could be, for example, sleep goggles or other devices close to the eye or head, such as headphones.

The invention is explained below by way of example with reference to the accompanying drawings. They show, schematically in each case,

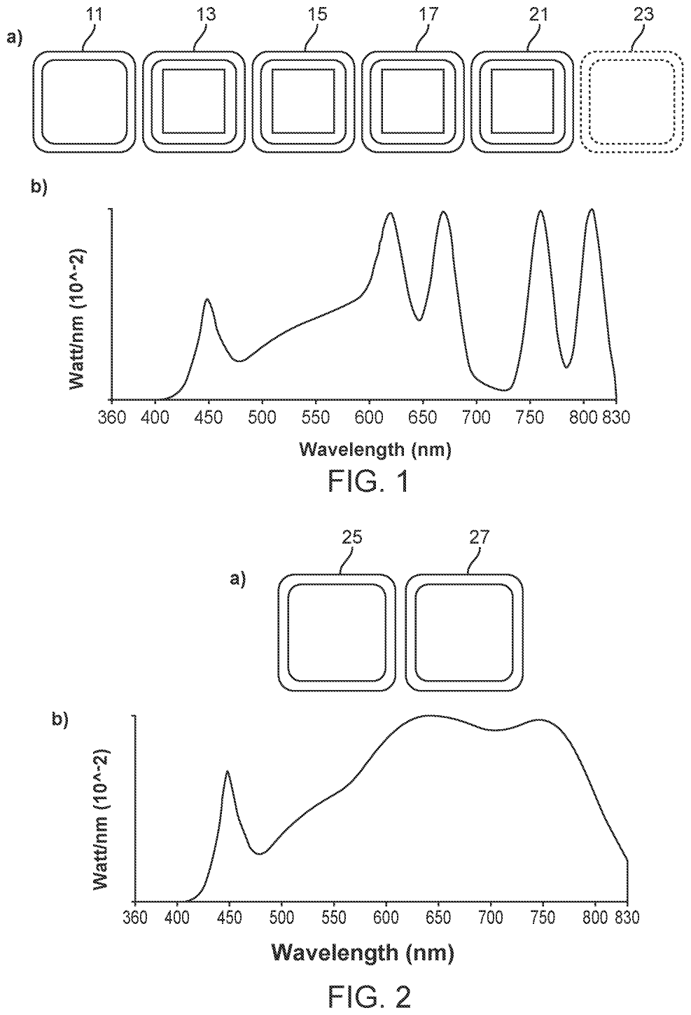

FIG. 1a a variant of an optoelectronic radiation device according to the invention,

FIG. 1b an emission spectrum similar to the radiation device of FIG. 1a,

FIG. 2a another variant of an optoelectronic radiation device according to the invention,

FIG. 2b an emission spectrum similar to the radiation device of FIG. 2a,

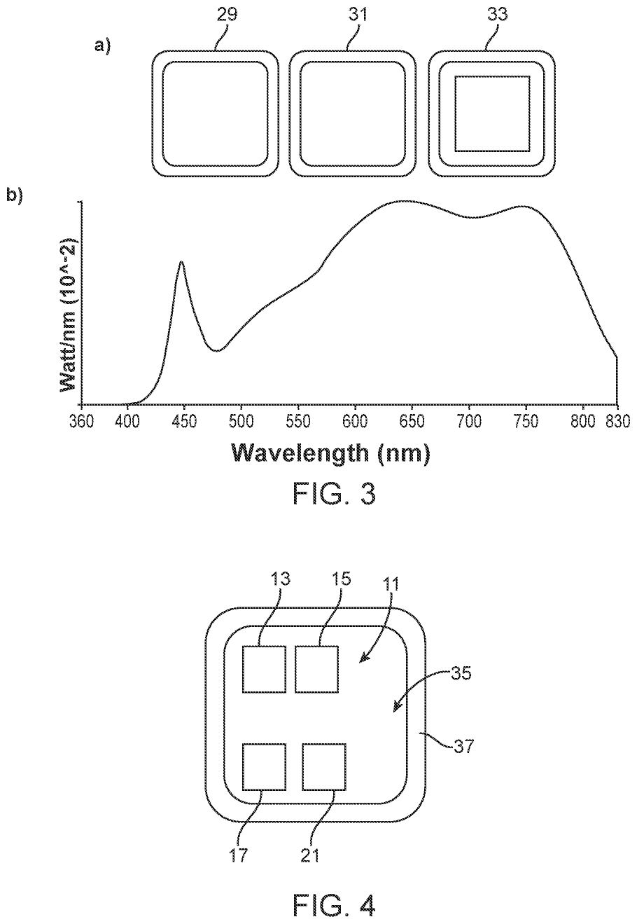

FIG. 3a another variant of an optoelectronic radiation device according to the invention,

FIG. 3b an emission spectrum similar to the radiation device of FIG. 3a,

FIG. 4 still another variant of an optoelectronic radiation device according to the invention,

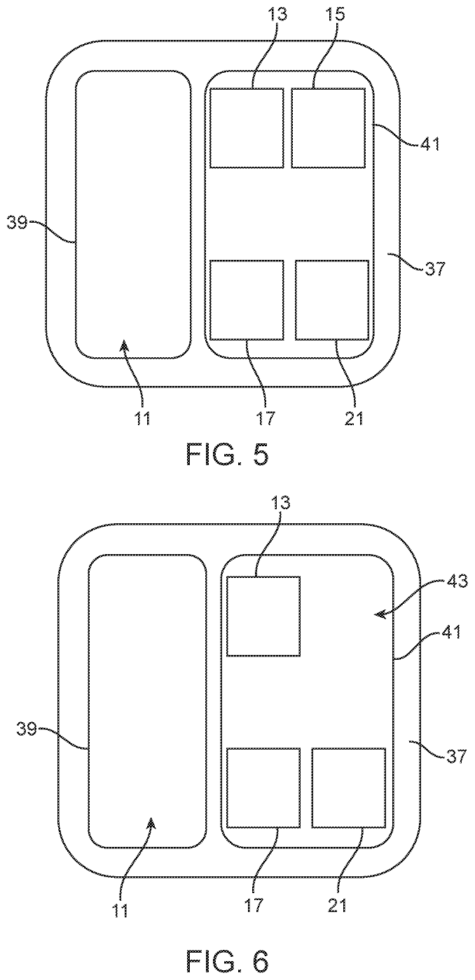

FIG. 5 still another variant of an optoelectronic radiation device according to the invention,

FIG. 6 yet another variant of an optoelectronic radiation device according to the invention,

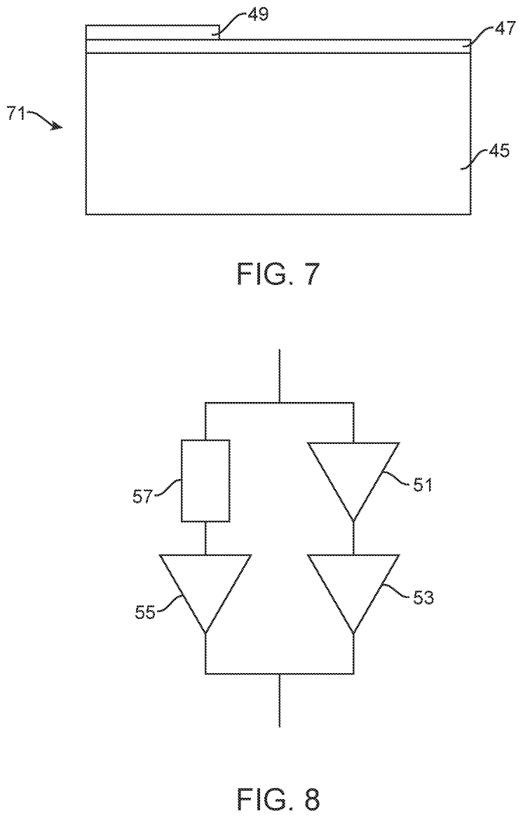

FIG. 7 a cross-sectional view of an optoelectronic radiation source that can be used in a variant of an optoelectronic radiation device according to the invention,

FIG. 8 a circuit diagram for the electrical connection of optoelectronic radiation sources according to a variant of an optoelectronic radiation device,

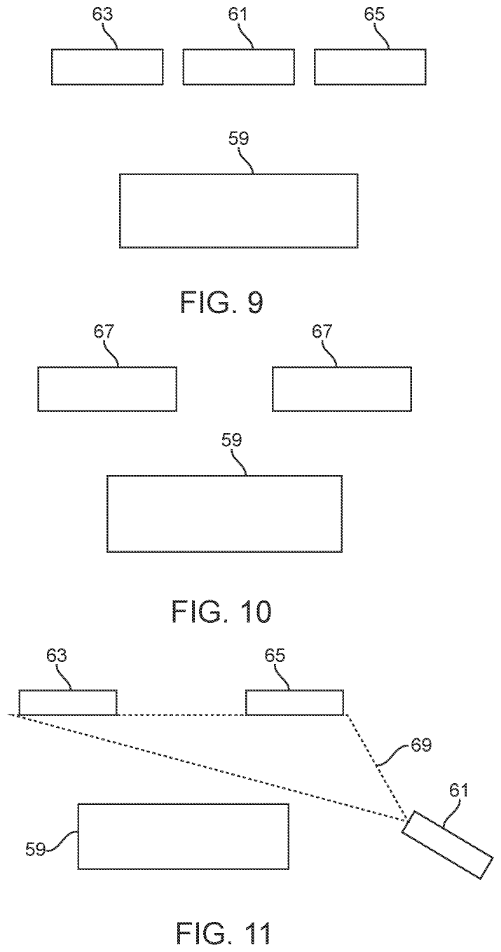

FIG. 9 a workplace scenario using a variant of an optoelectronic radiation device according to the invention,

FIG. 10 another workplace scenario, and

FIG. 11 yet another workplace scenario.

In the following figure description, the wavelengths λ1 to λ4 are referred to. The same applies to the wavelengths λ5 to λ8 or another selection of wavelengths, e.g., from the wavelengths λ1 to λ8. However, in some embodiments, wavelengths other than the wavelengths λ1 to λ4 can also be generated. In particular, the generated infrared radiation can be in the infrared spectral range between 600 nm and 900 nm or 600 nm and 860 nm. The spectrum may be continuous or interrupted.

The optoelectronic radiation device shown in FIG. 1a comprises five optoelectronic radiation sources 11, 13, 15, 17 and 21 and optionally a sensor 23. The optoelectronic radiation source 11 is configured to generate and emit white light, in particular in the spectral range from 430 nm to 650 nm. For this purpose, the optoelectronic radiation source can comprise an LED which generates blue light which, by means of a suitable conversion material known per se, generates white light, in particular with spectral components in the mentioned wavelength range.

The optoelectronic radiation source 13 is configured to generate infrared radiation at a wavelength of λ1=670 nm±10 nm. The optoelectronic radiation source 15 is configured to generate infrared radiation at a wavelength of λ2=760 nm±10 nm, the optoelectronic radiation source 17 is configured to generate infrared radiation at a wavelength of λ3=780 nm±10 nm, and the optoelectronic radiation source 21 is configured to generate infrared radiation at a wavelength of λ4=830 nm±10 nm.

FIG. 1b shows an approximate emission spectrum of the optoelectronic radiation device of FIG. 1a. In addition to visible spectral components, a continuous IR spectrum extends from components still visible to the eye at about 600 nm beyond the 830 nm shown in the scale of FIG. 1b to about 860 nm. The spectrum can also be considered a broad IR spectrum provided by the optoelectronic radiation device in addition to a visible light component in the wavelength range between about 420 nm and 630 nm.

The intensity of the emitted radiation is plotted against the wavelength. The emission spectrum is also continuous in the infrared spectral region up to above 860 nm, although the wavelengths are plotted only up to 830 nm. Strong changes in intensity over wavelength occur with high intensities at least approximately at wavelengths λ1, λ2, and λ3. Since the wavelength scale ends at 830 nm, a fourth “peak” at about wavelength λ4 is no longer plotted. Due to the continuous spectrum in the infrared spectral range, it is obvious that the radiation sources 13 to 21 can also emit infrared radiation that has other wavelengths than the mentioned wavelengths λ1 to λ4.

By generating and emitting white light by means of the optoelectronic radiation source 11, the optoelectronic radiation device can be used as an illumination device, for example in a room. In addition to the illumination function, the optoelectronic radiation device also provides a function for increasing well-being and/or health, in particular due to the infrared component in the radiated radiation. In particular, scientific studies have shown that spectral components at, for example, infrared wavelengths λ1, λ2, λ3, and λ4 can contribute to the promotion of health and regeneration of cell structure in the eye.

The sensor 23 may optionally be provided and configured to detect infrared radiation at at least one of the wavelengths λ1, λ2, λ3, and λ4 (or other wavelength). The sensor may be, for example, an LED that is operated in the blocking direction so that it acts as a sensor or a detector and can detect infrared radiation, particularly at one of the corresponding wavelengths λ1, λ2, λ3, or λ4.

The infrared radiation emitting radiation sources 13 to 21 may be intrinsically equipped to generate infrared radiation. In particular, each of the radiation sources may be an LED configured to generate infrared radiation at the respective wavelength λ1 to λ4 in its respective active zone. Alternatively, at least one of the infrared radiation sources 13 to 21 may also be configured to generate short-wave radiation, such as blue light, which is converted into infrared radiation via a suitable converter.

The five radiation sources 11 to 21 and the sensor 23 may be arranged in the optoelectronic radiation device in different ways. For example, each optoelectronic radiation source 21 may be housed in a separate housing or enclosure. It is also conceivable that each of the radiation sources 11 to 21 is arranged in a separate cavity of a housing of the optoelectronic radiation device (not shown). A respective light source 11 to 21 is thereby arranged on the bottom of a cavity. The upper side of the cavity distant from the bottom of the cavity may be closed by means of a window transparent to the electromagnetic radiation generated. The same can apply to the optional sensor 23.

The optoelectronic radiation device shown in FIG. 2a comprises an optoelectronic radiation source 25 for generating white light, in particular in the spectral range between 430 nm and 650 nm, and an optoelectronic radiation source 27 for generating a broad spectrum in the infrared wavelength range, in particular in the wavelength range between 650 nm and 850 nm. In this case, the infrared radiation comprises spectral components, for example, at the wavelengths λ1 to λ4 already mentioned above.

The optoelectronic radiation source 25 for generating white light may comprise an LED, in particular a blue light emitting LED chip. Furthermore, the radiation source 25 may comprise a converter material suitable for converting the blue light into white light, in particular with a continuous spectrum between 430 nm and 650 nm.

The light source 27 may be configured to generate infrared radiation by conversion. For example, the radiation source 27 may comprise an LED or LED chip to generate short wavelength light, such as blue light. Using a converter material known per se from the group of gallium oxides, a broad continuous spectrum in the infrared spectral range with wavelengths between 650 nm and 850 nm, as shown in FIG. 2b, can be generated.

The radiation source 27 may, alternatively or additionally, comprise so-called quantum dots for generating the infrared radiation. These can be manufactured with respect to the desired emission wavelengths, for example to one of the wavelengths λ1 to λ4. Thus, a spectrum can be generated in the infrared spectral range which, in contrast to the spectrum of FIG. 2b, comprises clearly protruding peaks at the emission wavelengths of the quantum dots.

The optoelectronic radiation device shown in FIG. 3a comprises an optoelectronic radiation source 29 for generating white light, in particular in the form of a continuous spectrum, with wavelengths between 430 nm and 650 nm.

In addition, the optoelectronic radiation device of FIG. 3a comprises an optoelectronic radiation source 31 for generating infrared radiation. This optoelectronic radiation source operates with a converter which is configured to convert light from a blue LED or ultraviolet radiation into infrared radiation with a continuous spectrum, for example between 650 nm and 850 nm.

The optoelectronic radiation source 33 is intrinsically configured to generate infrared radiation, for example at wavelength λ4. The radiation source 33 may be, for example, an LED chip that is intrinsically capable of generating infrared radiation at wavelength λ4. Tuning to other wavelengths, for example λ1, λ2 or λ3, is also possible.

The use of an optoelectronic radiation source 31 for generating a relatively broad infrared spectrum in combination with a further radiation source 33 for generating infrared radiation at a specific wavelength has the advantage that, for example, the dose of the radiation emitted by the radiation source 33 can be specifically adjusted and controlled. This is possible in particular if the radiation device according to FIG. 3a also uses a sensor as shown by way of example in FIG. 1a. Via the sensor, a detection of the radiation dose of the radiation emitted, for example, by the radiation source 33 is possible. By means of a control system not shown, the radiation source 33 can be controlled in such a way that it emits light depending on the radiation dose detected by means of the sensor.

The optoelectronic radiation device of FIG. 4 comprises, like the radiation device of FIG. 1a, a radiation source 11 for generating and emitting white light in the spectral range from 430 nm to 650 nm. It also comprises four optoelectronic radiation sources 13 to 21 for generating and emitting infrared radiation. The radiation source 13 is configured to emit infrared radiation at the wavelength λ1. Radiation source 15 is configured to emit infrared radiation at wavelength λ2, radiation source 17 is configured to emit infrared radiation at wavelength λ3, and radiation source 21 is configured to emit infrared radiation at wavelength λ4.

All radiation sources 11 to 21 are accommodated in a single cavity 35 in the radiation device of FIG. 4. The cavity 35 is formed in a housing 37 of the radiation device. The arrangement of all radiation sources in a single cavity 35 has the advantage that the optoelectronic radiation device can be made particularly compact.

A substrate can also be used instead of a cavity.

In contrast to the variant of FIG. 4, in the radiation device of FIG. 5 the optoelectronic radiation source 11 for generating white light is arranged in a separate first cavity 39, while the optoelectronic radiation sources for generating infrared radiation at the wavelengths λ1 to λ4 already mentioned above are accommodated in a further, second cavity 41. The first cavity 39 and the second cavity 41 are again formed in the housing 37 of the optoelectronic radiation device. A “cross-talk” between the radiation source 11 and the radiation sources 13 to 21 can be easily avoided due to the arrangement in different cavities.

In contrast to the variant of FIG. 5, the optoelectronic radiation device of FIG. 6 lacks the radiation source 15 for generating infrared radiation at the wavelength λ2. Instead, an optoelectronic radiation source not shown is provided, which emits shorter-wavelength light, such as blue light. In addition, a converter 43 is arranged in the cavity 41 to absorb the short wavelength light and convert it into infrared radiation. The generated infrared radiation may be broadband and, in particular, may include spectral components at wavelength λ2.

Variations on the variant according to FIG. 6 are conceivable. For example, one (or more) of the radiation sources 13 to 21 can be accommodated in a separate cavity. The radiation source for the emission of short-wave light with the converter material 41, which is not shown, can also be accommodated in a separate cavity. The accommodation in separate cavities has the advantage that thereby a “cross-talk” with other radiation sources can be avoided in a simple way.

The optoelectronic radiation source 71 shown in cross-section in FIG. 7 comprises a substrate 45 on which a first layer 47 is arranged. The first layer 47 is an epitaxial layer for generating blue light which is emitted in particular upwards. The formation of such a layer on a substrate for generating blue light is known per se. This layer can be formed from several thinner, individual layers, in particular for forming an active zone.

A second layer 49 is formed over a portion of the first layer 47, which is at least one epitaxial layer for generating infrared radiation, e.g., at one of the wavelengths λ1 to λ4. This epitaxial layer can be optically excited by the radiation from the first layer 47 and in turn generate and emit IR radiation. It is also possible for the entire first layer 47 to be covered by the second layer 49. Partial coverage has the advantage that the blue light from the first layer 47 is still available for conversion to white light. This conversion can be done by means of a conversion material arranged over the second layer 49 (not shown). The optoelectronic radiation source of FIG. 7 therefore fulfills a kind of double function, since both IR light and white light can be generated.

The circuit diagram shown in FIG. 8 illustrates one possibility for the electrical connection of optoelectronic radiation sources 51, 53, 55, which may be LEDs or LED chips. The radiation sources 51 and 53 for generating infrared radiation are connected in series. The current through the LEDs or LED chips can thus be kept constant, in particular by means of a current control element 57, such as a series resistor. The radiation source 55 for generating white light, in particular via conversion from blue or UV light, is connected in series with the current control element 57. The radiation source 55 and the current control element 57 are connected in parallel with the radiation sources 51 and 53, as shown in FIG. 8.

A voltage drop of 1.9 volts can occur at radiation source 51 and a voltage drop of 1.8 volts can occur at radiation source 53, while a voltage drop of 3 volts occurs at radiation source 55 and a voltage drop of 0.7 volts occurs at current control element 57. Different voltage drops at the different radiation sources can thus be compensated by a parallel connection and the use of series resistors or current control elements.

FIG. 9 illustrates a workplace scenario for illuminating a workstation 59 in a room or the like. A variant of an optoelectronic radiation device 61 according to the invention, which is configured to emit an IR broad spectrum according to the above explanations, can be arranged on the ceiling of the room in addition to lamps 63, 65 emitting white light. This allows a person working at the workplace to be exposed to the IR broad spectrum.

In the scenario shown in FIG. 10, two optoelectronic radiation devices 67 according to the invention, which can emit both white light and infrared radiation (see the spectra in FIGS. 1b, 2b and 3b), can be arranged on the ceiling above a workstation 67. Thus, not only white light but also infrared radiation can be provided at the workstation 67.

In the scenario of FIG. 11, in contrast to the scenario of FIG. 9, the optoelectronic radiation device 61 is not arranged on the ceiling next to the lamps 63, 65, but away from the ceiling and, for example, on the floor of the workspace. The optoelectronic radiation device 61 is arranged and designed in such a way that the generated infrared radiation is emitted in the form of a radiation cone 69. This radiation cone 69 floods the room and illuminates the ceiling of the room, as FIG. 11 illustrates.

### REFERENCE LIST

11 optoelectronic radiation source
13 optoelectronic radiation source
15 optoelectronic radiation source
17 optoelectronic radiation source
21 optoelectronic radiation source
23 sensor
25 optoelectronic radiation source
27 optoelectronic radiation source
29 optoelectronic radiation source
31 optoelectronic radiation source
33 optoelectronic radiation source
35 cavity
37 housing
39 cavity
41 cavity
43 converter
45 substrate
47 layer
49 layer
51 optoelectronic radiation source
53 optoelectronic radiation source
55 optoelectronic radiation source
57 current control element
59 workplace
61 optoelectronic radiation device
63 lamp
65 lamp
67 optoelectronic radiation device
69 light cone
71 optoelectronic radiation source

1. Optoelectronic radiation device, in particular for generating and emitting health-promoting and regenerative radiation, comprising
at least one or more optoelectronic radiation sources configured to generate infrared radiation, and
at least one optoelectronic radiation source for generating white light,
wherein the infrared radiation is in an infrared spectral range between 600 nm and 900 nm, and
wherein each radiation source is arranged in an associated, own cavity of a housing of the radiation device, or
at least two radiation sources are arranged in the same cavity or on a same substrate.

2. Optoelectronic radiation device according to claim 1,
characterized in that
the infrared radiation comprises spectral components at at least one and preferably at several or all of the following wavelengths:
λ1=670 nm±10 nm or ±30 nm
λ2=760 nm±10 nm or ±30 nm
λ3=780 nm±10 nm or ±30 nm
λ4=830 nm±10 nm or ±30 nm.

3. Optoelectronic radiation device according to claim 1,
characterized in that
the infrared radiation comprises spectral components at at least one and preferably at several or all of the following wavelengths:
λ5=620 nm±10 nm or ±30 nm
λ6=675 nm±10 nm or ±30 nm
λ7=760 nm±10 nm or ±30 nm
λ8=830 nm±10 nm or ±30 nm

4. Optoelectronic radiation device according to claim 2,
characterized in that
one, several or all of said wavelengths form a local maximum in the infrared emission spectrum of the radiation device.

5. Optoelectronic radiation device according to claim 1,
characterized in that
the device comprises a separate optoelectronic radiation source for generating infrared radiation at a predetermined wavelength, in particular at at least one, some and preferably at each of said wavelengths.

6. Optoelectronic radiation device according to claim 3,
characterized in that
one, several or all of said wavelengths form a local maximum in the infrared emission spectrum of the radiation device.

7. Optoelectronic radiation device according to claim 1,
characterized in that
at least one of the radiation sources, several of the radiation sources or each of the radiation sources is arranged as a separate radiation source, in particular with an own enclosure, on a carrier.

8. Optoelectronic radiation device according to claim 1,
characterized in that
at least one radiation source is intrinsically configured to generate infrared radiation, in particular at at least one of the wavelengths λ1 to λ4 or λ5 to λ8.

9. Optoelectronic radiation device according to claim 1,
characterized in that
at least one radiation source is intrinsically configured to generate radiation in a spectral range outside the infrared spectral range, in particular outside the wavelengths λ1 to λ4 or λ5 to λ8, and
that at least one converter is provided for at least partially converting the generated radiation into radiation comprising spectral components in the infrared spectral range, in particular at at least one of the wavelengths λ1 to λ4 or λ5 to λ8.

10. Optoelectronic radiation device according to claim 1,
characterized in that
at least one optoelectronic radiation source is configured both for generating white light and for generating infrared radiation, which comprises spectral components in the infrared spectral range, in particular at at least one of the wavelengths λ1 to λ4 or λ5 to λ8.

11. Optoelectronic radiation device according to claim 10,
characterized in that
the at least one optoelectronic radiation source comprises a converter which allows conversion of the radiation provided by the radiation source into white light and into infrared light.

12. Optoelectronic radiation device according to claim 1,
characterized in that
the at least one optoelectronic radiation source for generating white light and the at least one or more of the optoelectronic radiation sources for generating infrared radiation are arranged or arrangeable distant from each other.

13. Optoelectronic radiation device according to claim 1,
characterized in that
the radiation source for generating white light is arranged in a separate cavity of a housing of the radiation device.

14. Optoelectronic radiation device according to claim 1,
characterized in that
that the radiation source for generating white light and at least one radiation source for generating infrared radiation are arranged in a common cavity of the housing or on a substrate of the radiation device.

15. Optoelectronic radiation device according to claim 1,
characterized in that
the device comprises at least one sensor for detecting infrared radiation, in particular at at least one of the wavelengths λ1 to λ8, wherein, preferably, one or more of the emitters of the radiation sources are used as sensor.

16. Optoelectronic radiation device according to claim 15,
characterized in that
the device comprises a controller which is configured to control at least one radiation source for generating infrared radiation as a function of a signal generated by means of the sensor.

17. Optoelectronic radiation device according to claim 15,
characterized in that
the sensor is arranged in a cavity of a housing of the radiation device, wherein, preferably, at least one of the optoelectronic radiation sources for generating infrared radiation is arranged in the cavity.

18. Optoelectronic radiation device according to claim 1,
characterized in that
at least two and preferably all radiation sources for generating infrared radiation are electrically connected in series.

19. Optoelectronic radiation device according to claim 1,
characterized in that
at least one and preferably all radiation sources for generating infrared radiation are electrically connected in parallel to at least one radiation source for generating white light.

20. Electronic device, such as a cell phone, tablet, computer screen, television, comprising a display, and
at least one optoelectronic radiation device according to claim 1, wherein the optoelectronic radiation device is integrated into the display.

21. Electronic device, such as a cell phone, tablet, computer screen, television, comprising.
a display, and
at least one optoelectronic radiation device according to claim 1, wherein the optoelectronic radiation device is integrated into the housing, in particular such that the user can be irradiated with infrared radiation during use of the device.

22. Electronic device according to claim 21,
characterized in that
the device comprises a proximity sensor which can be used to generate infrared radiation in at least one of the wavelength ranges mentioned.

23. Motor vehicle with:
an interior and
at least one optoelectronic radiation device according to claim 1,
wherein the radiation device is arranged in the interior.
